(19) United States
(12) Patent Application Publication
(10) Pub. No.: US 2018/0030408 A1
(43) Pub. Date: Feb. 1, 2018

(54) Culture Medium for In-Vitro Cultivation of Mycobacterium Leprae (M. Leprae)

(71) Applicants: Debaprasad Pal, Kolkata (IN); Kumarjyoti Ghosh, Rahara (IN); Atanu Ray, Siliguri (IN)

(72) Inventors: Debaprasad Pal, Kolkata (IN); Kumarjyoti Ghosh, Rahara (IN); Atanu Ray, Siliguri (IN)

(21) Appl. No.: 15/661,207

(22) Filed: Jul. 27, 2017

(30) Foreign Application Priority Data
Jul. 27, 2016 (IN) 201631025713

(51) Int. Cl. C12N 5/00; G01N 33/569; C07K 14/35; A61K 39/04

(52) U.S. Cl. CPC C12N 5/0018; A61K 39/04; G01N 33/5695; C07K 14/35; A61K 31/00

(57) ABSTRACT

Provided herein is a culture medium for in-vitro cultivation of Mycobacterium leprae (M. Leprae) comprising: a) a solid phase comprising Lowenstein-Jensen medium, and b) a liquid phase comprising a stock solution of Middlebrook 7H9, Middlebrook Growth Supplement, thyroxine, glycerol and a mixture of an antifungal agents and antibiotics.

### CROSS-REFERENCE TO RELATED APPLICATION

This application claims priority to Indian Patent Application No. 201631025713 filed Jul. 27, 2016, the disclosure of which is hereby incorporated in its entirety by reference.

### FIELD OF THE INVENTION

This invention relates to a culture medium for in-vitro cultivation of Mycobacterium leprae (M. leprae).

This invention further relates to an in-vitro cultivation method for Mycobacterium leprae in said medium, said medium being a biphasic culture medium, and will be referred to hereinafter as PRG Medium.

### BACKGROUND OF THE INVENTION

Mycobacterium leprae was the first organism described as the causative agent of an infection. It was described by Dr. G. A. Hansen as the causative agent of leprosy in 1874. Thereafter, numerous agents/bacteria have been described as causative agents of disease like Mycobacterium tuberculosis, Vibrio cholera, etc. All bacteria discovered till now have been grown in artificial culture in chemically defined media (in-vitro cultivation) in the laboratory. However, Mycobacterium leprae though first described as a causative agent still remains uncultivable in the laboratory in artificial culture media. It is thus the only organism which does not fulfill the Koch's Postulate as it does not grow in artificial culture media as required, which is essential in labeling any organism as the causative agent of a disease.

Numerous attempts have been made by different workers to grow this organism in the laboratory but all attempts at cultivation have been unsuccessful till date. In 1880, Hillairet (Hillairet and Gaucher E. N Zote sur le parasitesme de la leprae. Compt. Revd. Soc. Biol. 2. 386, 1880) reported growing M. leprae in the laboratory, but later proved unsuccessful. In 1900, Kedrowsky (Kedrowsky W. J. Artificial culture of the leprosy bacilli. Russ. Arch. Pathol. Klin. Medi. Backteriol 10: 449, 1900) described an acid fast organism which later proved to be a saprophyte. Duval (Duval C. W. The cultivation of the leprosy bacillus and the experimental production of leprosy in the Japanese dancing mice. J. Exp. Biol. 12:649, 1910) used albumin, but grew only saprophytes. Shiga (Shiga K. Die Kultur and die kolo mieu bildung der lepra bagillen J. Chosen. Med Assoc. 19:541, 1929) treated lepra tissue with sulphuric acid but was again unsuccessful. Petroff's media was tried by McKinley & Soule (McKinley E. and Soule M H. Studies on leprosy experimental lesions in monkeys and cultivation of bacillus leprae J. Au. Medl. Assoc. 98:361, 1932) using different oxygen & CO₂ tension and mixed chick embryo in tyrode solution but Dharmendra & Lowe (Dharmendra & Lowe J. Attempts to cultivate M. leprimurium. IJMR 25; 835, 1938) failed to repeat the work. Lowenstein (Lowenstein E. The cultivation of the leprosy bacilli. Prelim. Communication. Int. J. Lepr. 3. 43; 1935) tried growing the organism in glycerine potato media, while Marchoux et al (Marchoux E., Chorine, V. Chanard I, issemil J. Negative leprosy transmission trials in hamsters. Ann. Inst. Pasteur 68:99, 1942) could not grow M. leprae in nutrient media with hens egg. Nakamura (Nakamura M. Studies on the cultivation of leprosy bacillus. HAWAII. Med. J. 8:265 1949) used a medium with added mucin from submaxillary gland of ox, but Nishimura & Honda (Nishimura S and Honda Supplementary experimentation on cultivation of Mycobacterium leprae and Mycobacterium leprae-murium by means of Nakamura Endo Mucin culture media La Lepra 18:109, 1949) failed to repeat the work. Bapat (Bapat C. V., Ranadive K. J., and Khanolkar V. R. Growth characteristic of an acid-fast mycobacterium isolated from human lepromatous leprosy. Int. J. Lepr 29.329.1961) grew (1949) acid fast organism in media containing fibroblasts from dried root ganglion of human fetus. This bacilli called ICRC bacilli however failed to grow in media without tissue culture. Baylet (Baylet R, Camain R, and Basset. Attempt to cultivate the Hansen bacilli on monkey kidney cells. Int. J. Lepr 29:542, 1961) tried to grow the organism in monkey kidney cell but failed but Delville (Delville J. P. Multiplication and behavior of Hansen bacilli in tissue culture. Int. J. Lepr. 31: 599, 1963) isolated diptheroids from Hansens lesions in Dubos broth enriched with 10% fetal calf serum. Chakraborty and Dastidar (Chakrabarty A. N., Dastidar S. G., Das S. and Chaudhury S. K. Repeated isolation of nocardia-like organisms from multibacillary cases of leprosy. Ind. J. Lepr 59:247.1987) reported isolation of nocardioform like organism from multibacillary cases of leprosy while Chatterjee B. R. (Chatterjee B. R. A non-acid fast coccoid precursor-possible cultivable phase of mycobacterium leprae. Lepr. India 48:398, 1976) produced evidence of non acid fast coccoid precursors of M. leprae which reverted back to mycobacterial forms in intraperitoneally injected twice. Mori (Mori T. Cultivation of Mycobacterium leprae on modified Ogawa yolk media. Jap. J. Lepr 46; 48, 1977) claimed success using 1% Ogawa yolk medium with DOPA L. cystein, and thioglycollate. Portacls and Pattyn (Portacls F. and Pattyn S. R. Isolation of fastidiously growing mycobacteria from armadillo livers infected with Mycobacterium leprae Int. J. Lepr, 50:370, 1982) claimed successful isolation from armadillo liver in Ogawa agar slant with added various mycobacteria, adjusted to various pH, temperature and CO₂. Veerarhaghavan (Veerarhaghavan N. Method of cultivation of M. leprae, Current sci 52; 1983) claimed isolation M. leprae in Medium V. at 4° C.-10° C., but turned out to be rapidly growing mycobacteria.

Therefore, there have been numerous unsuccessful attempts at cultivating this organism on artificial culture media. The International Leprosy Congress 1978 concluded that “so far there is no proof that a genuine culture of the leprosy bacilli has been obtained”. This position holds good even today. Therefore, the need exists to provide a medium for the growth of Mycobacterium leprae in a rapid and cost-effective manner.

### OBJECTS OF THE INVENTION

It is therefore an object of this invention to propose a culture medium for in-vitro cultivation of Mycobacterium leprae.

It is a further object of this invention to propose a culture medium for in-vitro cultivation of Mycobacterium leprae, which leads to a rapid growth of M. leprae within 12-16 weeks.

Another object of this invention is to propose a culture medium for in-vitro cultivation of Mycobacterium leprae, which is cost-effective as well as to reduce generation time.

Yet another object of this invention is to propose a culture medium for in-vitro cultivation of Mycobacterium leprae, which is easy to prepare, using easily available standard materials.

These and other objects of the invention will be apparent to a reader on reading the ensuing description, in conjunction with the accompanying drawings.

### BRIEF DESCRIPTION OF THE ACCOMPANYING DRAWINGS

FIG. 1: Z.N. Stain Smear from PRG Medium showing solidly stained acid fast bacilli.

FIG. 2: Pearly white colonies of PRG Medium at 16 weeks.

FIG. 3: Globi formation in culture typical of Mycobacterium leprae (Z.N. Stain).

FIG. 4: Gel image showing the amplified repetitive gene of 29 base pair of Mycobacterium leprae.

### BRIEF DESCRIPTION OF THE INVENTION

Thus according to this invention is provided a culture medium for in-vitro cultivation of Mycobacterium leprae (M. leprae) (PRG Medium).

According to this invention is further provided an in-vitro cultivation method for M. leprae using said medium.

In accordance with this invention, the PRG Medium is a biphasic medium comprising

a) Solid phase comprising standard Lowenstein-Jensen medium

b) Liquid phase comprising a stock solution of Middlebrook 7H9, Middle-brook Growth Supplement, thyroxine, glycerol, and at least one selected from an antifungal agent and antibiotic.

The Middlebrook 7H9 broth base used for the stock solution has the composition:

| Ingredients | Grams/litre |
|---|---|
| Ammonium sulphate | 0.50 |
| Disodium phosphate | 2.50 |
| Monopotassium phosphate | 1.00 |
| Sodium citrate | 0.10 |
| Magnesium sulphate | 0.05 |
| Calcium chloride | 0.0005 |
| Zinc sulphate | 0.001 |
| Copper sulphate | 0.001 |
| Ferric et ammonium citrate | 0.04 |
| L-glutamic acid | 0.50 |
| Pyridoxine | 0.001 |
| Biotin | 0.0005 |

The Middlebrook ADC Growth Supplement is used as an enrichment supplement for the cultivation of Mycobacteria and has the following composition:

| Formulae | (per vial sufficient for 500 ml media) | |
|---|---|---|
| Bovine Albumin | 2.50 | g |
| Dextrose | 1.00 | g |
| Catalase | 0.001 | g |
| Distilled Water | 50 | ml |

The process for the preparation of the culture medium comprises the steps of providing a solid phase comprising Lowenstein-Jensen medium (L.J.Medium) and providing a liquid phase comprising a stock solution. The stock solution is prepared by dissolving Middlebrook 7H9 broth base in distilled water followed by sterilization to obtain a sterile medium, cooling the sterile medium to a temperature of about 40° C.-50° C. and aseptically adding 1 vial of. Middlebrook growth supplement thereto, followed by adding thyroxine sodium and dissolving, by adding sterile glycerol slowly to the medium and antifungal agents and antibiotics, to obtain the stock liquid medium. The stock liquid medium is added to the solid medium by passing it through a sterile millipore filter to avoid any contamination. This constitutes the culture medium (PRG Medium) which is stored at 2° C.-8° C. and can be used within 4-6 weeks.

About 2-4 gms Middlebrook 7H9 is added to 400 to 500 ml water and the solution is sterilized by autoclaving for about 15-20 mins. The thyroxine sodium can be added in any form, preferably in the form of a tablet and about 20 to 60 microgram (mcg) is added to the sterile medium.

The antibiotics are selected from chloramphenicol and aminoglycosides such as gentamicin, amikacin and the like. The antibiotics are added in a proportion of 400 to 600 mg per 400 to 500 ml of the sterile medium.

The antifungal agents are selected from amphotericin and azoles such as Itraconazole, ketoconazole etc. The antifungal agents are added in 50 to 200 mg per 400 to 500 ml of the sterile medium.

In accordance with this invention is further provided, an in vitro cultivation method for M. leprae in the artificial culture media that is developed.

Tissue taken from the ear lobule or from the lesions of lepromatous leprosy patients which were positive on smear examination, and graded 4+ to 6+ as per standard grading classification given, were inoculated into the PRG medium in duplicate. Thereafter, the incubation was done in duplicate at 18° C.-20° C. and also at 37° C. Incubation of the media was carried out in a slightly slanting position so that the liquid portion covers the solid portion of the media. Incubation was carried out for a total period of 12-16 weeks before declaring the culture as negative. Details about the composition of the PRG Media are described below:

It was observed that solidly stained acid fast bacilli as stained by modified Ziehl-Neelsen's stain resembling Mycobacterium leprae appeared in the liquid phase at about one month's time. Thereafter, colonies started to appear on the solid phase at about 6-8 weeks time gradually attaining full size at about 12-16 weeks. Colonies appeared only in the PRG Media incubated in the B.O.D incubator at 18° C.-22° C. No colony formation was observed at 37° C. even after 12-16 weeks. The colonies that appeared in the PRG media in the B.O.D. incubator were 2-3 mm pearly white in colour, occasionally with a buff centre and rough. They were non-emulsifiable in nature.

This invention will now be explained in greater details with the help of the following non-limiting examples:

### EXAMPLE

### 1) Composition of the Culture Medium (BI-Phasic PRG Media)

The culture medium according to the invention (PRG MEDIA) is a bi-phasic media consisting of a solid and a liquid phase.

A) SOLID PHASE: The solid phase consisted of standard Lowenstein-Jensen media (L. J. Media) as marketed commercially.

B) LIQUID PHASE: A stock solution of the liquid phase was prepared as follows: Middlebrook 7H9 marketed commercially was added as follows:

1) 2.35 gm in 450 ml distilled water was dissolved and sterilized by autoclaving at 15 lbs at 121° C. for 15-20 minutes.

2) This sterile media was cooled to about 45° to 50° C. and 1 vial of Middlebrook Growth Supplement as available commercially was added aseptically to this. The supplement was at first warmed to 45° to 50° C. and shaken well to make a uniform suspension 1 vial of the supplement is added to 450 ml of sterile, molten, cooled (45-50° C.) Middlebrook 7H9 Broth base, the solution is mixed well, poured in sterile bottle and sealed with a cap.

3) Thyroxine sodium tablet of 50 mcg was added to the above mixture and dissolved.

4) Glycerol 2 ml sterilized separately and cooled to 45° C. was added slowly to the above mixture aseptically.

5) Antifungal and antibiotic mixture was added to the above mixture as follows:

a) Chloramphenicol—500 mg

b) Gentamicin—40 mg (1 ml)

c) Amphotericin B—50 mg dissolved in 10 ml sterile distilled water

d) Itraconazole—100 mg

The procedure was carried out in a Safety Cabinet Level- 2. This constituted the Stock Liquid Media which was stored at 2° C.-8° C. until further use.

### 2) Preparation of the Complete PRG Media

Stock solution of the liquid phase (2-3 ml) was added to the solid phase (L. J. Media) by passing it through sterile membrane filter (0.22 U) Millipore type to avoid any contamination. This constituted the complete PRG Media which was stored at 2° C.-8° C. and used up within 4-6 weeks.

The medium was used for in-vitro cultivation of M. leprae obtained from patients affected with leprosy.

### 3) Selection of Patients/Collection of Material

Lepromatous leprosy patients attending the Skin O. P. D. of M. G. M. Medical College, Kishanganj, Bihar were selected for study. Consent was taken from them for collection of the material. The skin over the ear lobe was aseptically cleaned with alcohol and iodine application. A slit-skin smear from the ear lobe or punch biopsy from nodules was taken. When nodules were present on the body, the skin was cleaned aseptically, pinched up, and a cut was made with a sharp scalpel. The material was collected over the scalpel blade and subsequently transferred onto a glass slide.

### 4) Smear Examination

Material collected as above was heat fixed on the glass slide and stained by modified Ziehl-Neelsen's stain. The Solutions required for modified Ziehl-Neelsen's stain are:

1) Carbol-fuchin

2) 5% Sulphuric acid

3) 0.5% Methylene blue

Method of staining:

A smear is made, allowed to dry and fixed by heat. The slide is flooded with hot carbol fuschin, reheated at intervals and stained for 10 minutes, washed in running water and decolourized in 5% sulphuric acid for 5-10 minutes till the film is faintly pink after washing. It is again washed in running tap water. Countered with 0.5% methylene blue for 1 minute again, washed in running tap water and allowed to dry.

Thereafter, smears were air dried, and examined under the microscope. Positive smears were graded according to standard grading method from 1+ to 6+. The grading of the smears was done according to WHO specifications:

1-10 bacilli in 100 fields=1+

1-10 bacilli in 10 fields=2+

1-10 bacilli per field=3+

10-100 bacilli per field=4+

100-1000 bacilli per field=5+

22 1000 bacilli, clump and globi in every field=6+

Those materials which were 4+ to 6+ positive were selected for culture in prepared bi-phasic PRG Media in duplicate.

### 5) Incubation of the Media

Collected material was teased to expose the inner content of the tissue and also cut into two pieces. One portion was incubated at 18-22° C. in the B. O. D. incubator and the other half was incubated at 37° C. Incubation at both temperatures was done in a slightly slanting position so the liquid phase covered the solid phase of the media. Incubation was carried out for a total period of 12-16 weeks.

### 6) Observation

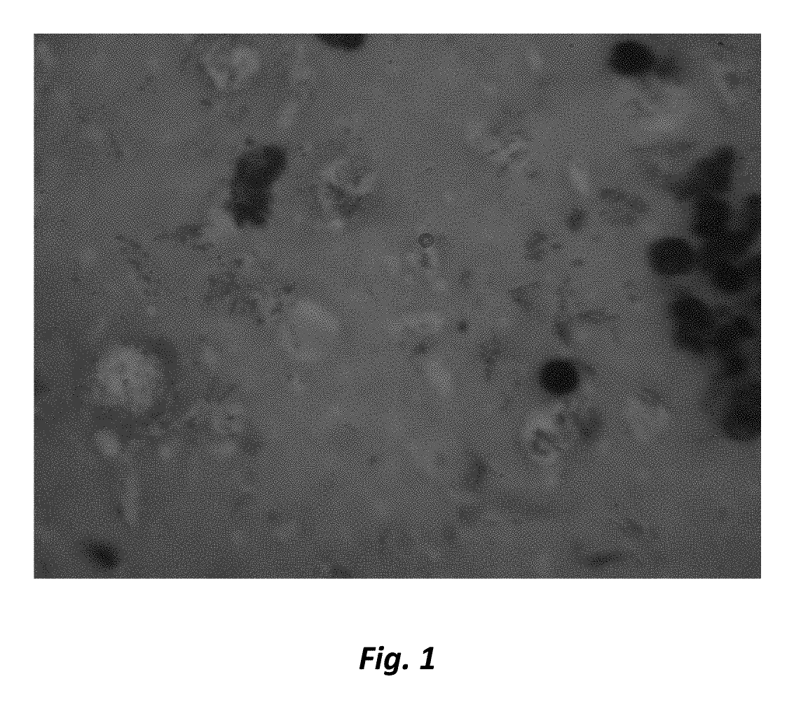

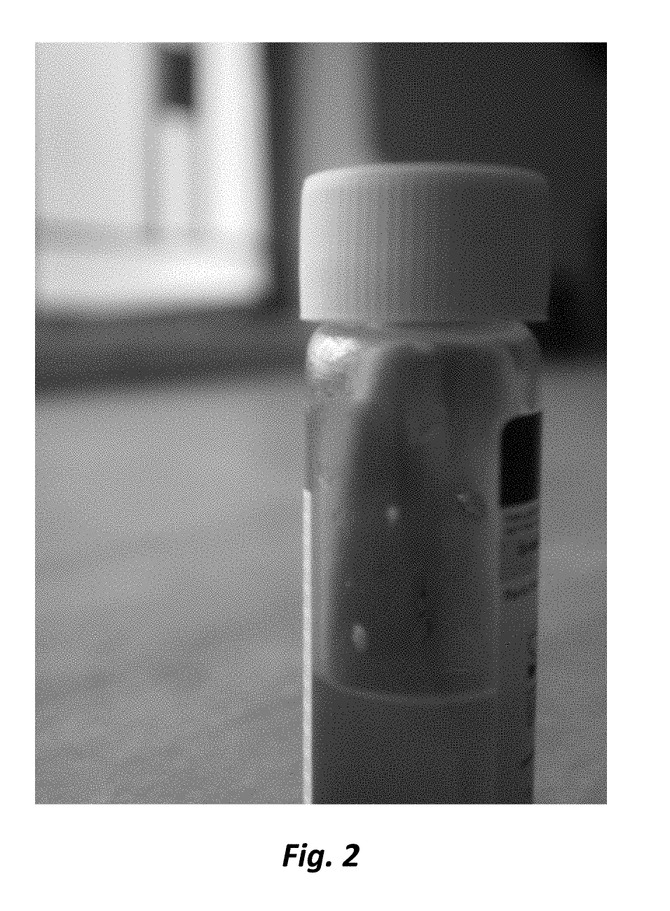

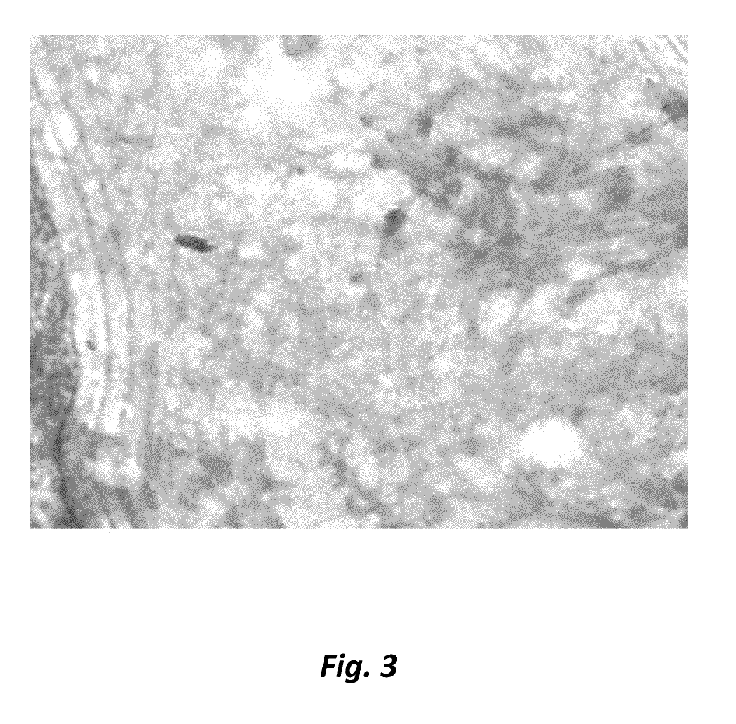

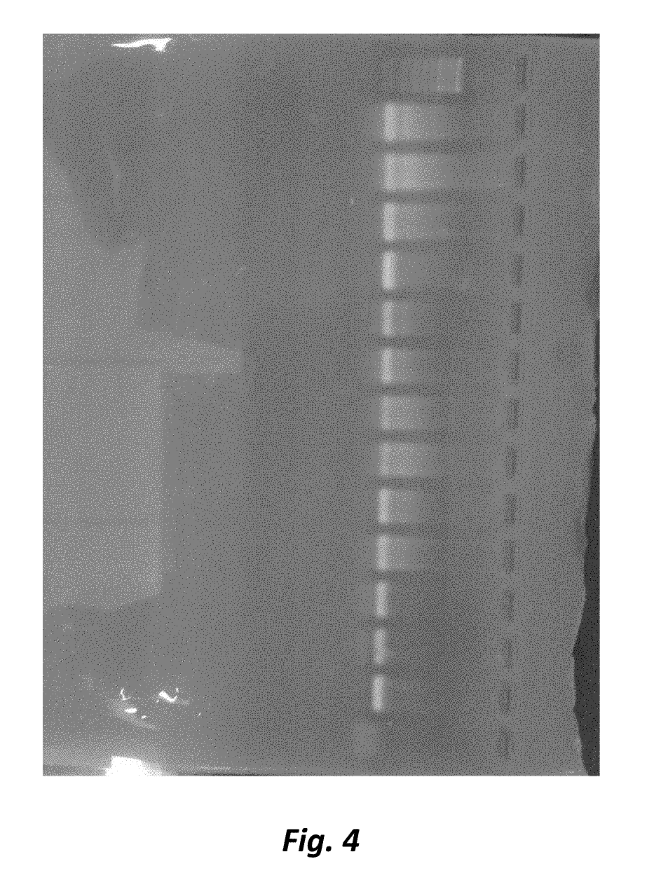

Organisms first grew in about 4-6 weeks in the liquid phase at 18° C. -22° C. No growth was observed at 37° C. Smears made from the liquid phase and stained by modified Ziehl-Neelsen's method showed solidly stained acid fast bacilli with globi formation (FIG. 1). No fragmented or beaded bacilli were observed. On further incubation at 18° C. -22° C., colonies started to appear in the solid phase at about 8-10 weeks and thereafter grew to their full size at about 12-16 weeks. Full grown colonies were 2-3 mm in diameter, pearly white in colour, occasionally with a buff centre (FIG. 2). Colonies were rough, difficult to pick up and largely non-emulsifiable. Smears made from these colonies and stained by modified Ziehl-Neelsen's stain showed similar solidly stained acid fast bacilli with globi formation (FIG. 3). No fragmented or beaded bacilli were observed. Colonies grown on PRG media was subjected to PCR test using RLEP gene. FIG. 4 shows the gel image showing the amplified repetitive gene of 129 base pair, the sequence of Mycobacterium leprae which is 100% specific to the species (JALMA Institute, Agra). Uptil now 100 biopsy specimens taken from lesions of fresh untreated lepromatous leprosy patients were cultured in Bi-phasic PRG Media developed by the workers and incubated at 18-22 C and at 37 C for a period of 16 weeks or longer if necessary.

Turbidity first appears in the liquid phase at about 6-8 weeks at 18-22 C, indicating growth in the liquid phase. Smears made from the liquid phase and stained by modified Z.N. stain showed acid fast bacilli with globi formation, which is strongly indicative of Mycobacterium leprae. On further incubation, colonies start to appear on the solid phase at around 10 to 12 weeks time, which attains full colony size by around 16 weeks or longer if required. Smears made from the solid phase and stained by Modified Z.N. stain showed similar morphology as in the liquid phase. Colonies that appeared in the solid phase were pearly white in character, 2-3 mm in diameter and largely non-emulsifiable. Out of 100 specimens put up for culture in the PRG media, 91 showed turbidity in the liquid phase and a similar number showed colony formation in the solid phase. No turbidity or colony formation was observed in cultures incubated at 37 C.

The inventors describe here the formulation of an artificial bi-phasic culture media consisting of a solid and a liquid phase in which Mycobacterium leprae which is acid fast has been repeatedly cultivated and isolated successfully in pure culture from infected tissue of leprosy patients.

1. A culture medium for in-vitro cultivation of Mycobacterium leprae (M. Leprae) comprising:
a) a solid phase comprising Lowenstein-Jensen medium,
b) a liquid phase comprising a stock solution of Middlebrook 7H9, Middlebrook Growth Supplement, thyroxine, glycerol and a mixture of an antifungal agent and an antibiotic.

2. The culture medium as claimed in claim 1, wherein said antibiotic is selected from the group consisting of chloramphenicol and aminoglycosides.

3. The culture medium as claimed in claim 1, wherein said antifungal agent is selected from the group consisting of amphotericin and azoles.

4. A process for the preparation of a culture medium for in-vitro cultivation of Mycobacterium Leprae (M. Leprae) comprising: providing a solid phase of Lowenstein-Jensen medium; preparing an aqueous solution of Middlebrook 7H9 broth base, followed by sterilization to obtain a sterile medium; cooling the sterile medium and aseptically adding thereto Middlebrook growth supplement, followed by thyroxine sodium, slowly adding glycerol, an antibiotic, and an antifungal agent to obtain a stock liquid medium; and adding said stock liquid medium to said solid phase by passing the stock liquid medium through a multipore filter, to obtain the culture medium.

5. The process as claimed in claim 4, wherein 2-4 gms Middlebrook 7H9 is added to 400 to 500 ml water and the solution is sterilized by autoclaving for 15-20 minutes to obtain the sterile medium.

6. The process as claimed in claim 4, wherein 20 to 60 mcg thyroxine sodium is added to the sterile medium.

7. The process as claimed in claim 4, wherein the contents of 1 vial of Middlebrook ADC Growth Supplement is added to 400-500 ml of the sterile medium.

8. The process as claimed in claim 4, wherein 400 to 600 mg of the antibiotic is added to 400 to 600 ml of the sterile medium.

9. The process as claimed in claim 4, wherein 50-200 mg of the antifungal agent is added to 400 to 500 ml of sterile medium.

10. A process for the in-vitro cultivation of M. Leprae using the culture medium as claimed in claim 1, wherein tissue from a patient having lepromatous leprosy is taken and inoculated into the culture medium, so that the liquid phase covers the solid phase of the medium, allowing incubation at a temperature in the range of 18-22° C., for a period of 12-16 weeks to obtain colonies of the bacteria, which on staining by Ziehl-Neelsen's method showed colonies of solidly stained acid fast bacilli with globi formation.

11. The process as claimed in claim 10, wherein said colonies are grown until they reach 2-3 mm in diameter, are pearly white in colour, occasionally with a buff centre, are rough, are difficult to pick up, and are largely non-emulsifiable.
